(19) United States
(12) Patent Application Publication
(10) Pub. No.: US 2002/0058338 A1
(43) Pub. Date: May 16, 2002

(54) Cell growth method and device with multiple applications

(76) Inventors: Luc Douay, Saint-Maur (FR); Jean-Luc Magalhaes, Boissy Sans Avoir (FR); Florence Hummel, Montesson (FR)

Correspondence Address: Lawrence E. Laubscher, Sr., EFS Customer No. 30267311, Suite 300, 745 South 23rd Street, Arlington, VA, 22202, US

(21) Appl. No.: 09/992,423

(22) Filed: Nov. 26, 2001

(30) Foreign Application Priority Data
May 26, 1999 (FR) 99-06820
May 16, 2000 (FR) PCT/FR00/01311

(51) Int. Cl. C12N 5/00

(52) U.S. Cl. 435/383

(57) ABSTRACT

A cell growth method and device with multiple applications, in particular to the maintenance, proliferation, amplification or differentiation of cells in a closed enclosure (10), including individual flow rate or quantity adjustment means (30, 36) for determining compositions and flow rates of mixtures of different nutrient media or constituents and compositions and flow rates of different mixtures of growth factors, corresponding to an envisaged application, and then to modify the compositions and/or the flow rates of the mixtures of nutrient media or constituents and of the mixtures of growth factors delivered to the enclosure, which modifications can be effected independently of each other.

The invention relates to a cell growth method and device with multiple applications, intended in particular, although not exclusively, for growing hematopietic cells (blood cells, hematopietic progenitors and stem cells), using a bioreactor, for example that described in the document EP-A-0474847.

Methods and devices of this type have already been thoroughly investigated because of their benefit in diverse applications, such as grafts, the production of particular cell types, etc.

International application WO96/40858 (AASTROM BIOSCIENCES INC.) discloses a device for growing biological cells, in particular hematopietic cells, including a cassette in which a growth chamber is formed, a container for a nutrient medium containing growth factors, a waste container for spent medium, a bag for harvesting cells, and means for interconnecting the above components to form a sterile closed system. The device is first used on a first instrument to fill the growth chamber with the nutrient medium, inoculate cells in the growth chamber, and distribute cells in the chamber by agitation, and then on a second instrument (an incubator) for two weeks, during which the growth chamber and the nutrient medium container are maintained at predetermined temperatures, the nutrient medium, oxygen and other gases are delivered into the device at predetermined flow rates, the temperature and flow rate parameters being defined to correspond to optimum conditions for cell growth, some of them being liable to vary in time in accordance with a predefined program.

The essential drawback of the above prior art device is its lack of adaptability and variability, as a consequence of which the device is “fixed” and intended for a specific application, in particular because:

the nutrient medium used is a predefined mixture that cannot be modified during application, containing growth factors that are diluted and distributed homogeneously in the mixture so that neither the constituents of the nutrient medium nor the quantity of growth factors that it contains can be modified after the start of the application,

it is impossible to adjust separately the flow rates of the nutrient medium and the growth factors delivered to the growth chamber,

the program that defines any variation of the nutrient medium flow rate is predefined (it is stored in a memory supplied with the device), and cannot be modified in operation or before starting an operation,

the composition and the flow rate of the gas mixture delivered to the device are predefined and cannot be varied during application,

the device is made in the form of a “kit” for a single, automated use, intended for a specific application and usable by non-specialist medical personnel who are required to follow the instructions to the letter, without taking any initiative, and

it is not possible to monitor the cell growth process during application.

The present invention has entirely different objectives.

It provides a method and a device for growing cells enabling modification at will and at any time of not only the flow rate but also the composition or the nature of a nutrient medium delivered to a growth chamber.

It also provides a method and a device of the above type that also enable modification at will and at any time of the composition and the flow rate of a mixture of growth factors delivered to the growth chamber, these modifications being independent of the aforementioned modifications of the composition and the flow rate of the nutrient medium.

It further provides a method and a device of the above type enabling modification at will and at any time of the composition and the flow rate of the ventilation gas mixture delivered to the growth chamber, these modifications being independent of the aforementioned modifications of the composition and the flow rate of the nutrient medium and the composition and the flow rate of the mixture of growth factors.

It further provides a method and a device of the above type suitable for a plurality of different applications, rather than a single and dedicated application.

It further provides a method and a device of the above type that can be used equally well as a research tool and for a clinical or other application, for example in a hospital environment to grow cells taken from a patient or a donor, with a view to subsequent reinjection.

It further provides a method and a device of the above type that, subject to minor modifications, can be used with growth chambers with different volumes, covering a range from approximately 1 milliliter to approximately 1 liter.

The invention proposes a method of growing cells, which method has multiple applications, in particular to the maintenance, proliferation, amplification or differentiation of cells in a closed enclosure, and consists of introducing cells into said enclosure, delivering to said enclosure ventilation gases, nutrient media and growth factors, and harvesting the cultivated cells, characterized in that it further consists of:

providing multiple sources of different nutrient media or constituents and multiple sources of different growth factors,

determining compositions and flow rates of mixtures of the different nutrient media or constituents and compositions and flow rates of mixtures of the different growth factors corresponding to an envisaged application, and then

during the progress of said application, modifying at regular or irregular intervals the compositions and/or the flow rates of the mixtures of nutrient media or constituents and the mixtures of growth factors delivered to the enclosure, which modifications can be effected independently of each other.

It has been found, surprisingly, that the quantity of growth factors delivered to a cell growth bioreactor is a sensitive parameter that is critical for cell expansion and that even relatively slight variations of this parameter about an optimum value have significant effects on cell expansion, which is considerably reduced, whereas the same variations have practically no influence on cell expansion in a static growth system (bag growth). It is therefore particularly important, in a continuous or discontinuous perfusion growth system such as a bioreactor, to be able to adjust precisely the quantities of growth factors delivered to the bioreactor. The invention enables these adjustments throughout the growth period, regardless of the type of application (maintenance, proliferation, amplification or differentiation of cells).

The same applies to the composition and the flow rate of the nutrient medium delivered to the bioreactor, which the invention allows to be varied and adjusted at will.

In particular, the nutrient medium can be delivered continuously or discontinuously.

On the one hand, this significantly increases the yield from a bioreactor and, on the other hand, it enables the same type of bioreactor to be used for different applications.

According to another feature of the invention, the method further consists of providing a plurality of different gases, determining a composition and a flow rate of a mixture of the different gases corresponding to said envisaged application, delivering said gas mixture to the enclosure and, if necessary, and at regular or irregular intervals, modifying the composition and/or the flow rate of the gas mixture delivered to the enclosure.

The facility to modify the gas mixture delivered to the enclosure is favorable in terms of increasing the yield from a bioreactor and the diversity of applications that can be envisaged.

According to yet another feature of the invention, the method further consists of periodically or non-periodically testing the development of the cells in said enclosure as the aforementioned application proceeds and modifying said compositions and/or flow rates as a function of the results of said tests.

These features of the method according to the invention enable the definition for each application of optimum conditions for delivery to a cell growth chamber and optimum variations of those conditions in time, checking of the progress of the application at periodic or non-periodic intervals, and, as a function of the result of such checking, modifying the delivery conditions and variations therein as much and as often as necessary.

This significantly improves the cell growth yield in the various applications that can be envisaged.

The invention proposes also a device for growing cells, which device has multiple applications, in particular to the maintenance, proliferation, amplification or differentiation of cells, and includes a closed enclosure, microporous membranes delimiting a growth chamber in said enclosure, means for delivering cells to said chamber, means for delivering ventilation gases, nutrient media and growth factors to the enclosure, and means for harvesting cells, characterized in that it further includes containers of different nutrient media or constituents connected by flow rate adjustment means to a delivery manifold of said enclosure, containers of different growth factors connected by flow rate adjustment means to another delivery manifold or the same delivery manifold of the enclosure, and means for controlling the aforementioned adjustment means to determine, as a function of an envisaged application, the composition and/or the flow rate of a mixture of nutrient media and the composition and/or the flow rate of a mixture of growth factors delivered to the enclosure via said delivery manifold or manifolds and to modify said compositions and/or said flow rates as said application proceeds.

Advantageously, the device includes containers for different gases, flow rate adjustment means connecting said containers to a delivery manifold of said enclosure, and means for controlling the adjustment means to determine the composition and the flow rate of a gas mixture delivered to said enclosure as a function of the envisaged application and to modify said composition and/or said flow rate if necessary as the application proceeds.

The invention will be understood better and other features, details and advantages thereof will become more clearly apparent on reading the following description, which is given by way of example and with reference to the accompanying drawings, in which:

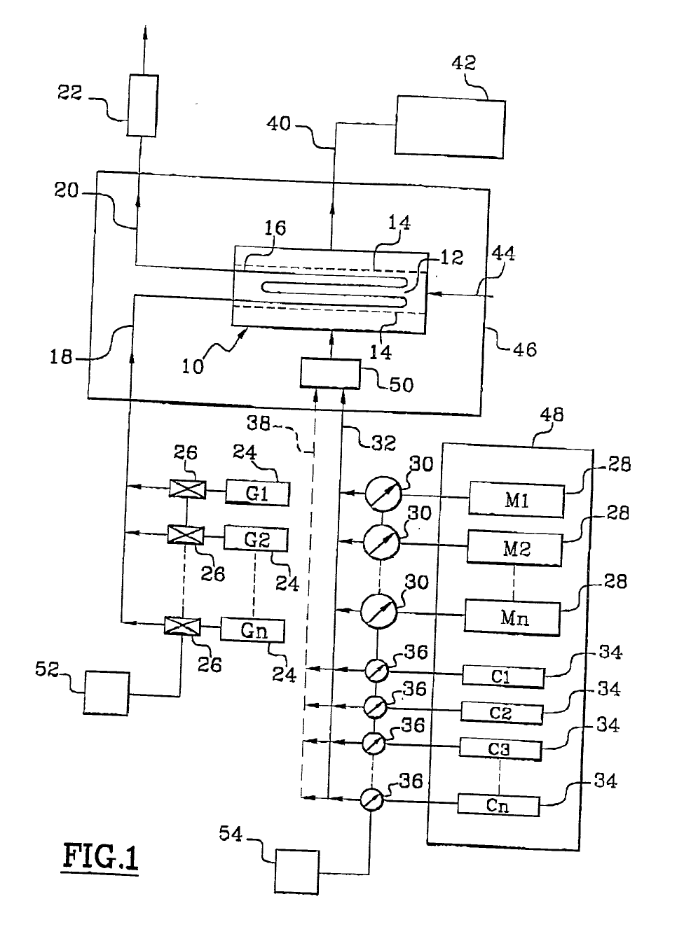

FIG. 1 is a diagrammatic view of a cell growth device according to the invention;

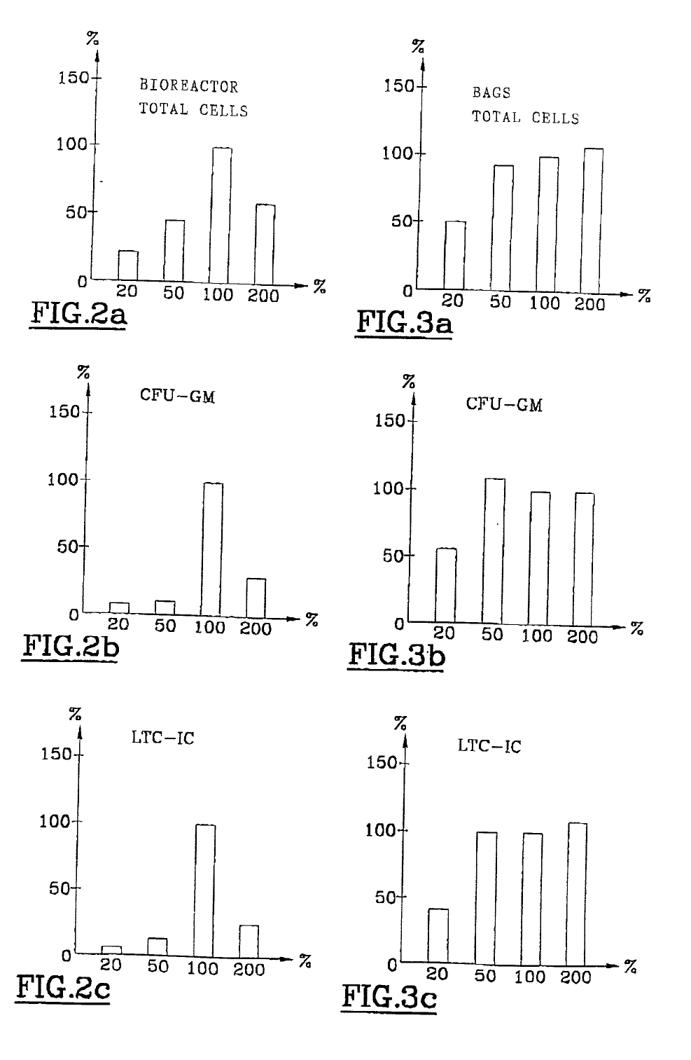

FIGS. 2a, 2b and 2c are graphs showing the effect of the quantity of growth factors on expansion of nucleated cells, granulomacrophagic progenitors and stem cells in a cell growth bioreactor; and

FIGS. 3a, 3b and 3c show the same influence in static bag growth.

The cell growth device shown diagrammatically in FIG. 1 is intended in particular, although not exclusively, for the growth, proliferation, differentiation and/or amplification of biological cells, in particular hematopietic cells, said device essentially including a bioreactor that is preferably of the type described in the document EP-A-0474847 or in the document PCT/FR98/02548, the contents of which are hereby incorporated by way of reference.

Essentially, the bioreactor (a detailed description of which can be found in the above documents) includes a disposable and closed enclosure 10 in which a growth chamber 12 is delimited by microporous membranes 14 having a pore size from approximately 0.1 to approximately 5 μm, forming barriers for confining hematopietic cells, and whose characteristics are determined as a function of the type of cells to be cultivated.

At least one layer of gas circulation capillary tubes 16 is disposed in the growth chamber 12, said tubes consisting of porous hollow fibers that are regularly distributed between the membranes 14 and are connected, on the one hand, at an inlet end, to a delivery manifold 18 in turn connected to gas delivery means and, on the other hand, at the outlet end, to an outlet manifold 20 connected to a container 22 for temporarily storing outgoing gases, which can be analyzed to monitor the progress of cell growth in the chamber 12.

In accordance with the invention, the gas delivery means include a plurality of containers 24 containing different pressurized gases G1, G2, . . . , Gn (for example oxygen, nitrogen, CO₂, etc.) and each of which is connected via individual flow rate adjustment means 26 to the delivery manifold 18.

The enclosure 10 is equipped with delivery means for nutrient medium and growth factors and extraction means for spent nutrient media.

The nutrient medium and growth factor delivery means include a plurality of containers 28 containing different nutrient media M1, M2, . . . Mn (or different nutrient medium constituents) and each connected by individual flow rate or quantity adjustment means 30 to a delivery manifold 32 which forms a mixer and which discharges into the enclosure 10 below the growth chamber 12 in the embodiment shown. The delivery means also include containers 34 containing different growth factors, for example different cytokines C1, C2, C3, . . . Cn, each container 34 being connected via individual quantity or flow rate adjustment means 36 to a delivery manifold, which can be the manifold 32 used to deliver nutrient media or a different manifold, such as that shown in dashed outline at 38, the manifold 38 discharging into the enclosure 10 below the growth chamber 12, like the manifold 32, or being connected to the latter upstream of the enclosure 10, possibly via a mixing chamber.

The quantities of growth factors used are very small, for which reason the growth factors are diluted in appropriate media, for example the aforementioned nutrient medium, so that the quantities or the flow rates to be delivered to the enclosure 10 can be adjusted more easily and more accurately.

A spent medium extraction manifold 40 connects the top part of the enclosure 10, above the growth chamber 12, to a container 42.

In some cases some of the spent medium extracted from the enclosure 10 via the manifold 40 can be filtered and reinjected into the enclosure, below the growth chamber 12.

A lateral wall of the enclosure 10 is equipped with means 44 (shown symbolically here by an arrow) for injecting or inoculating cells into the growth chamber 12 and sampling or harvesting cultivated cells in the chamber 12, said means 44 being of a type known in the art for injecting cells into the growth chamber 12 or sampling cells from it in a sterile manner.

The enclosure 10 is placed inside a thermostatically controlled enclosure 46 which is adjusted to the required growth temperature and outside which are located the gas, nutrient medium and growth factor delivery means and the extracted gas container 22 and spent medium container 42. The containers 28 containing the nutrient media or their constituents and the containers 34 containing the growth factors are placed inside a refrigerated enclosure 48.

Outside the thermostatically controlled enclosure 46, the delivery manifold or manifolds discharge into a buffer container 50 which forms a degassing and temperature adjustment chamber and is connected to the delivery arrangement for the enclosure 10, enabling introduction into the growth chamber of a nutrient medium and growth factors at the temperature required for cell growth (for example 37° C.).

The aforementioned means 26 for adjusting the flow rate of the various gases G1, G2, . . . , Gn are controlled valves of a type known in the art for adjusting the pressure and the flow rate of each gas in the delivery manifold 18.

The means 30 and 36 for adjusting the flow rates or quantities of nutrient media and growth factors introduced into the delivery manifold or manifolds 32, 38 are controlled micropumps such as peristaltic pumps which preserve the sterility of the nutrient media and can operate over extensive ranges of flow rates, the nutrient medium flow rate possibly varying from approximately 1 times to approximately 4 times the volume of the growth chamber per day, which corresponds to flow rates of approximately 0.1 to 0.5 ml/h for a 3 ml growth chamber, for example, the nutrient media and growth factors being delivered continuously or discontinuously. Controlled injection means of the syringe pusher type can equally be used as delivery means.

Control means 52, 54, which are advantageously computer-controlled, are associated with the various flow rate or quantity adjustment means 26, 30, 36 described above, on the one hand, for predefining optimum conditions for delivering to the enclosure 10 ventilation gases, nutrient media and growth factors, as well as optimum variations in time of the delivery conditions, and, on the other hand, for modifying the delivery conditions and variations therein as a function of the results of any tests on the progress of a growth within the chamber 12 carried out at periodic or non-periodic intervals. The tests can be carried out either using the means 44 for sampling cells in the growth chamber 12 or, without intrusion into the growth chamber 12, using optical means of a type known in the art.

The multiple facilities for adjusting the compositions, quantities or flow rates of gases, nutrient media and growth factors delivered to the enclosure 10 mean that the device according to the invention can be adapted to diverse applications, for example cell maintenance (consisting of keeping cells alive for a certain time period in vitro), proliferation (cell multiplication phase), amplification (increase in the number of cells as a consequence of proliferation), and differentiation (obtaining mature cells having a specific type and function).

For example, a mixture of a nutrient medium and a plurality of cytokines can be delivered to the growth chamber, the delivery flow rate can be varied in accordance with a predefined program and/or as a function of tests on the progress of cell growth, after which another nutrient medium suitable for conservation of the cultivated cells is delivered to the growth chamber. The nutrient medium can also be changed while cell growth is proceeding, etc.

These multiple adjustment facilities can also optimize the operation and the yield of the bioreactor, in particular through precise adjustment of the quantities of growth factors used, as these are very costly products and their concentrations are a highly sensitive and critical factor of cell growth in a bioreactor.

This has been verified, firstly by experiments to maintain mononucleated medullary cells for thirteen days at a concentration of 10⁵ cells/ml in a growth medium containing a mixture of six different cytokines (experiments A) and in an identical growth medium containing no cytokines (experiments B). The results (which are averages for three experiments) are set out in the table below and are expressed in terms of expansion factors relative to day 0 after three days of amplification.

| | Expansion factor | | |
|---|---|---|---|
| | Cells | BFU-e | CFU-GM |
| Experiments A | 4.71 ± 1.38 | 1.24 ± 0.75 | 9 ± 4.2 |
| Experiments B | 0.12 ± 0.06 | 0.08 ± 0.04 | 0.24 ± 0.12 |

BFU-e = erythroid progenitors
CFU-GM = granulomacrophagic progenitors

The role of the growth factor concentration was highlighted in comparative trials to grow cells, on the one hand, in a bioreactor (i.e. in a growth medium with continuous or discontinuous perfusion of nutrient medium and growth factors), and, on the other hand in bags (i.e. in a static medium); the curves in FIGS. 2 and 3 show the results of these experiments.

In these figures, the concentration of cytokines as a percentage of an optimum concentration corresponding to the value 100 is plotted on the abscissa axis. The expansion of the total nucleated cells, the CFU-GM granulomacrophagic progenitors, and the LCT-IC stem cells, respectively, as a percentage of the expansion obtained for the optimum concentration of cytokines corresponding to the value 100, is plotted on the ordinate axis.

The total nucleated cells include mature cells, progenitors and stem cells.

Comparing FIGS. 2a and 3a, showing the expansion of the total cells, indicates that the expansion is relatively insensitive to the concentration of cytokines when it varies from 50 to 200% of the optimum concentration in a static growth system (FIG. 3a) and is halved in a bioreactor if the concentration of cytokines is respectively 50% or 200% of the optimum concentration (FIG. 2a).

The same observations apply to the expansion of the CFU-GM cells (FIGS. 2b and 3b) and the LTC-IC cells (FIGS. 2c and 3c).

The experiments whose results are represented in FIGS. 2 and 3 were carried out with a mixture of six different cytokines, for which the maximum expansions of total nucleated cells, CFU-GM cells and LTC-IC cells were obtained in a bioreactor, for example with a concentration of 100 ng of three cytokines, 5 ng of a fourth cytokine, and 10 ng of a fifth and a sixth cytokine. FIGS. 2a, 2b and 2c show that, when the concentration of cytokines was less than the optimum value, the expansion of the total cells, the CFU-GM cells and the LTC-IC cells was a function of the quantity of cytokines and that beyond the optimum concentration value expansion was sharply reduced.

In the embodiment shown in FIG. 1, the enclosure 10 features a “front” delivery system for nutrient media and growth factors. It could instead feature a tangential filtered delivery system, as described in the document EP-A-0474847, including a layer of tubes with walls permeable to the nutrient media.

1. A method of growing cells, which method has multiple applications, in particular to the maintenance, proliferation, amplification or differentiation of cells in a closed enclosure (10), and consists of introducing cells into said enclosure, delivering to said enclosure ventilation gases, nutrient media and growth factors, and harvesting the cultivated cells, characterized in that it further consists of:
providing multiple sources (28) of different nutrient media or constituents and multiple sources (34) of different growth factors,
determining compositions and flow rates of mixtures of the different nutrient media or constituents and compositions and flow rates of mixtures of the different growth factors corresponding to an envisaged application, and then
during the progress of said application, modifying at regular or irregular intervals the compositions and/or the flow rates of the mixtures of nutrient media or constituents and the mixtures of growth factors delivered to the enclosure (10), which modifications can be effected independently of each other.

2. A method according to claim 1, characterized in that it further consists of providing a plurality of different gases, determining a composition and a flow rate of a mixture of the different gases corresponding to said envisaged application, delivering said gas mixture to the enclosure (10) and, if necessary, and at regular or irregular intervals, modifying the composition and/or the flow rate of the gas mixture delivered to the enclosure.

3. A method according to claim 1, characterized in that it further consists of periodically or non-periodically testing the development of the cells in said enclosure (10) as the aforementioned application proceeds and modifying said compositions and/or flow rates as a function of the results of said tests.

4. A method according to claim 1, characterized in that nutrient medium is delivered to the enclosure (10) continuously or discontinuously.

5. A method according to claim 1, characterized in that the aforementioned cells are hematopietic cells.

6. A device for growing cells, which device has multiple applications, in particular to the maintenance, proliferation, amplification or differentiation of cells, and includes a closed enclosure (10), microporous membranes (14) delimiting a growth chamber (12) in said enclosure, means (44) for delivering cells to said chamber, means for delivering ventilation gases, nutrient media and growth factors to the enclosure, and means (44) for harvesting cells, characterized in that it further includes containers (28) of different nutrient media or constituents connected by flow rate adjustment means (30) to a delivery manifold (32) of said enclosure, containers (34) of different growth factors connected by flow rate adjustment means (36) to another delivery manifold or the same delivery manifold (38, 32) of the enclosure, and means (52) for controlling the aforementioned adjustment means (30, 36) to determine, as a function of an envisaged application, the composition and/or the flow rate of a mixture of nutrient media and the composition and/or the flow rate of a mixture of growth factors delivered to the enclosure via said delivery manifold or manifolds and to modify said compositions and/or said flow rates as said application proceeds.

7. A device according to claim 6, characterized in that said manifold or manifolds (32, 38) are connected to said enclosure (10) by a degassing and temperature adjustment chamber (50).

8. A device according to claim 6, characterized in that it includes containers (24) for different gases, flow rate adjustment means (26) connecting said containers to a delivery manifold (18) of said enclosure, and means (50) for controlling the adjustment means (26) to determine the composition and the flow rate of a gas mixture delivered to said enclosure as a function of the envisaged application and to modify said composition and/or said flow rate if necessary as the application proceeds.

9. A device according to claim 6, characterized in that said cells are hematopietic cells.
